(19) United States
(12) Patent Application Publication
(10) Pub. No.: US 2005/0137629 A1
(43) Pub. Date: Jun. 23, 2005

(54) Trended measurement of cardiac resynchronization therapy

(76) Inventors: John A. Dyjach, Circle Pines, MN (US); Jeffrey E. Stahmann, Ramsey, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/730,760

(22) Filed: Dec. 8, 2003

(51) Int. Cl. A61N 1/362; A61B 5/04

(52) U.S. Cl. 607/9; 600/510

(57) ABSTRACT

One aspect relates to a method to provide trended measurement of cardiac resynchronization therapy (CRT). In various embodiments of the method, data related to a status of a prescribed cardiac resynchronization therapy (CRT) in a cardiac rhythm management (CRM) device is recorded, and the data is processed into trended data useful for assessing the status of the prescribed CRT. The trended data is presented for use to assess the status of the prescribed CRT and assist with determining programming adjustments for improving the prescribed CRT. Other aspects are provided herein.

### TECHNICAL FIELD

This application relates generally to cardiac rhythm management and, more particularly, to methods, devices and systems to provide trended measurement of cardiac resynchronization therapy.

### BACKGROUND

Cardiac rhythm management (CRM) devices are implantable devices that provide electrical stimulation to selected chambers of the heart in order to treat disorders of cardiac rhythm. An implantable pacemaker, for example, is a CRM device that paces the heart with timed pacing pulses. The most common condition for which pacemakers are used is in the treatment of bradycardia, where the ventricular rate is too slow. Atrio-ventricular conduction defects (i.e., AV block) that are permanent or intermittent and sick sinus syndrome represent common causes of bradycardia for which permanent pacing may be indicated. If functioning properly, the pacemaker makes up for the heart's inability to pace itself at an appropriate rhythm in order to meet metabolic demand by enforcing a minimum heart rate.

Also included within the concept of cardiac rhythm is the manner and degree to which the heart chambers contract during a cardiac cycle to result in the efficient pumping of blood. For example, the heart pumps more effectively when the chambers contract in a coordinated manner. The heart has specialized conduction pathways in both the atria and the ventricles that enable the rapid conduction of excitation (i.e., depolarization) throughout the myocardium. These pathways conduct excitatory impulses from the sino-atrial node to the atrial myocardium, to the atrio-ventricular node, and thence to the ventricular myocardium to result in a coordinated contraction of both atria and both ventricles. This both synchronizes the contractions of the muscle fibers of each chamber and synchronizes the contraction of each atrium or ventricle with the contralateral atrium or ventricle. Without the synchronization afforded by the normally functioning specialized conduction pathways, the heart's pumping efficiency is greatly diminished. Patients who exhibit pathology of these conduction pathways, such as bundle branch blocks, can thus suffer compromised cardiac output.

Heart failure refers to a clinical syndrome in which an abnormality of cardiac function causes a below normal cardiac output that can fall below a level adequate to meet the metabolic demand of peripheral tissues. Heart failure may present itself as congestive heart failure (CHF) due to the accompanying venous and pulmonary congestion. Heart failure can be due to a variety of etiologies with ischemic heart disease being the most common. Some heart failure patients suffer from some degree of AV block or are chronotropically deficient such that their cardiac output can be improved with conventional bradycardia pacing. Such pacing, however, may result in some degree of uncoordination in atrial and/or ventricular contractions because pacing excitation from a single pacing site is spread throughout the myocardium only via the much slower conducting muscle fibers of either the atria or the ventricles, and not the specialized conduction pathways. Most pacemaker patients can still maintain more than adequate cardiac output with artificial pacing, but the diminishment in cardiac output may be significant in a heart failure patient whose cardiac output is already compromised. Intraventricular and/or interventricular conduction defects are also commonly found in heart failure patients and can contribute to cardiac dysfunction by causing unsynchronized contractions during intrinsic beats.

In order to treat these problems, CRM devices have been developed which provide electrical pacing stimulation to one or more heart chambers in an attempt to improve the coordination of atrial and/or ventricular contractions, termed cardiac resynchronization therapy (CRT). As currently implemented, CRT has few mechanisms to adapt therapy as the patient's condition changes. Changes to the patient's condition may result in loss of degradation of the currently programmed CRT, also referred to herein as prescribed CRT, for a patient. In addition, changes to the patient's condition may be transient and not present while a physician is present. Thus, it is difficult to improve a CRT prescription for a given patient.

### SUMMARY

The above-mentioned problems are addressed by the present subject matter and will be understood by reading and studying the following specification. Various aspects and embodiments of the present subject matter maintain and report a historical data record of various parameters related to successful delivery of a prescribed CRT therapy. A physician is able to access meaningful information to assess the chronic, ambulatory status of prescribed CRT, and thus is able to improve the prescribed CRT.

One aspect relates to a method to provide trended measurement of CRT or parameters associated with CRT. In various embodiments of the method, data related to a status of a prescribed CRT in a CRM device is recorded, and the data is processed into trended data useful for assessing the status of the prescribed CRT. The trended data is presented for use to assess the status of the prescribed CRT and assist with determining therapy adjustments for improving the prescribed CRT or other heart failure therapy.

One aspect relates to an implantable CRM device. According to various embodiments, the CRM device comprises a plurality of interface channels to interface with a plurality of electrodes on at least one lead, a memory, a controller, and a communication circuit. The plurality of interface channels are adapted to deliver pacing pulses to at least one of the plurality of electrodes and to receive sensed cardiac signals from at least one of the plurality of electrodes as part of a prescribed CRT. The controller communicates with the plurality of interface channels and with the memory to control delivery of the pacing pulses, to process the sensed signals, and to record CRT-related data relevant to a status of the prescribed CRT to the memory. The communication circuit transmits the CRT-related data to an external device for presentation of data trends useful to assess status of the prescribed CRT.

One aspect relates to a system. According to various embodiments, the system comprises an implantable CRM device to perform a prescribed CRT and a programmer to program the CRM device to provide the prescribed CRT. The CRM device includes means for delivering pacing pulses to at least one of a plurality of electrodes, means for receiving sensed cardiac signals from at least one of the plurality of electrodes, means for controlling delivery of the pacing pulses and processing the sensed cardiac signals to perform the prescribed CRT, means for recording CRT-related data corresponding to a status of the prescribed CRT, and means to transmit and receive wireless communication signals. The programmer includes means to transmit and receive wireless communication signals such that the programmer is capable of wirelessly communicating with the CRM device, and means to display information corresponding to trended data indicative of the status of the prescribed CRT. Various system embodiments include a repeater and/or an advanced patient management (APM) device that are capable of wirelessly communicating with the CRM device, and/or are capable of displaying and/or processing information corresponding to trended data indicative of the status of the prescribed CRT.

This Summary is an overview of some of the teachings of the present application and not intended to be an exclusive or exhaustive treatment of the present subject matter. Further details about the present subject matter are found in the detailed description and appended claims. Other aspects will be apparent to persons skilled in the art upon reading and understanding the following detailed description and viewing the drawings that form a part thereof, each of which are not to be taken in a limiting sense. The scope of the present invention is defined by the appended claims and their equivalents.

### BRIEF DESCRIPTION OF THE DRAWINGS

FIG. 1 illustrates a block diagram of a multi-site pacemaker configurable to deliver CRT, according to various embodiments of the present subject matter.

FIG. 2 illustrates a display of marker/interval data, according to various embodiments of the present subject matter.

FIG. 3 is a flowchart illustrating a triggered data storage method, according to various embodiments of the present subject matter.

FIG. 4 illustrates a system that includes an implantable CRM device, a programmer, and an optional portable advance patient management (APM) device, according to various embodiments of the present subject matter.

FIG. 5 illustrates an implantable CRM device such as can be used in the system of FIG. 4, according to various embodiments of the present subject matter.

FIG. 6 illustrates a programmer or a portable APM device such as can be used in the system of FIG. 4, according to various embodiments of the present subject matter.

FIG. 7 illustrates a display such as can be projected on the programmer of FIG. 4, according to various embodiments of the present subject matter.

FIG. 8 is a flow diagram illustrating a trending method according to various embodiments of the present subject matter.

FIG. 9 provides a graph illustrating a trend example according to various embodiments of the present subject matter.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. This description references signal transmission lines using labels, and to simplify the discussion, also references the signals transmitted on the signal transmission lines using the same labels. The following detailed description is, therefore, not to be taken in a limiting sense, and the scope is defined only by the appended claims, along with the full scope of legal equivalents to which such claims are entitled.

Cardiac rhythm management devices for delivering cardiac resynchronization therapy (CRT) may be programmed with a number of different parameter settings that affect the manner in which resynchronization paces are delivered. These parameters can be initially programmed after implantation while a physician is monitoring the patient. During the initial programming, the physician analyzes the efficacy of the prescribed CRT and adjust the programming accordingly. However, the patient's condition may change subsequently, resulting in a loss or degradation of resynchronization therapy if the parameters are unaltered. Also, the operating characteristics of the device may change after implantation, either due to changes in the device itself or environmental influences, and that may also interfere with the optimal delivery of resynchronization therapy. Both of these situations may arise transiently and not be present when the patient is being evaluated during clinical follow-up. Thus, compared to the prescribed CRT evaluated by a physician in a clinical setting, the chronic, ambulatory status of the prescribed CRT may have a reduced efficacy in treating comprised cardiac output.

CRT delivery involves providing timed pacing stimuli to one or more pacing sites to improve coordination in the contraction of the heart. Successful CRT delivery involves delivering pacing pulses at desired site(s) with desired timing with respect to other cardiac events. For example, one CRT configuration synchronizes the right ventricle (RV) and left ventricle (LV) through appropriate RV and LV pacing, and is capable of functioning in a tracking mode (atrial tracking) and in a non-tracking mode. In this example, successful CRT involves delivering ventricular pacing pulse(s) to the prescribed ventricular site(s), delivering the ventricular pacing pulse(s) at the prescribed delay from the preceding atrial event when functioning in a tracking mode, and delivering ventricular pacing pulse(s) at the prescribed inter-site delay(s) when the prescribed CRT includes pacing at more than one site. The present subject matter is not limited to CRT with particular pacing sites or CRT operating in a particular mode. The teaching provided in this disclosure can be applied to other prescribed CRT involving other cardiac pacing sites and modes.

The efficacy of an originally-prescribed CRT may be lost or degraded for a number of reasons, including but not limited to the following examples: the loss or reduction of left ventricle (LV) and right ventricle (RV) pacing; the loss or reduction of LV pacing but not RV pacing; and the reduction of synchronization effectiveness with no loss of ventricular pacing pulses.

There may be one or more reasons for the loss or reduction of LV and RV pacing. For example, the PR interval (the period of time from the onset of the P wave, i.e. atrial depolarization, to the onset of the QRS complex, i.e. ventricular depolarization) may be too short with respect to the programmed AV delay (the period of time between the atrial sensed or paced pulse, and delivery of ventricular pulses). Another possible reason is that the programmed maximum tracking rate (MTR) is too low, the programmed maximum sensing rate (MSR) is too low, and/or the programmed maximum pacing rate (MPR) is too low. Another possible reason is that the programmed refractory windows are incorrect. A refractory window is a period of time with a predetermined duration and predetermined delay after an event, such as a sensed or paced event. Another possible reason is that the sensor response is too low when the CRM device is operating in a VVI(R) (ventricular inhibited pacing with rate modulation) mode. Another possible reason is that programmed ventricular rate regulation (VRR) is too low. VRR is referenced in U.S. Pat. No. 6,285,907, for example. The VRR feature is designed to improve the delivery of resynchronization therapy in the presence of atrial arrhythmias. Another possible reason is that a programmed lower rate limit (LRL) for pacing is too low when the CRM device is operating in the VVI(R) mode. Another possible reason involves environmental RV noise.

There may be one or more reasons for the loss or reduction of LV pacing but not RV pacing. For example, the programmed LVPP (the LV protection period) may be too long. LVPP is referenced in U.S. patent application Publication 2002/0082655, for example. Another possible reason is that the PR interval may be too short with respect to the programmed AV delay and programmed LV offset, which is a programmed time window from a RV event in RV-based timing. Another possible reason is that the intrinsic RV-LV interval changed because of, for example, the progression of heart disease and the resulting pathology of conduction pathways. Another possible reason involves environmental and/or far field LV noise.

There may be one or more reasons for the reduction of synchronization effectiveness with no loss of ventricular pacing pulses. For example, the myocardial substrate may have changed, because of, for example, the progression of heart disease such that the conduction of excitation changed. Another possible reason is the loss of capture. Capture represents an electrical discharge sufficient to successfully depolarize and contract a cardiac chamber. For example, a one-to-one capture occurs when each pacemaker output pulse results in a contraction of the appropriate chamber. Another possible reason involves atrial pacing rather than ventricular pacing, which can cause poor synchronization between the left atrium (LA) and the LV. Another possible reason involves a sensed AV delay offset. The AV delay offset corresponds to and is intended to compensate for the difference in conduction speed between an intrinsic and paced signal.

It would be useful for a physician to have data representing the status of the CRT, such as historical or chronic status of the CRT away from a clinical setting, in order to assist in the diagnosis and correction of problems causing compromised therapy. In accordance with various aspects of the subject matter within this disclosure, an implantable cardiac rhythm management device is configured to store certain data representative of the chronic, ambulatory status. The stored data includes data sensed from implanted or external sensors and other data, such as paced data, operating mode, time, and various programmed parameters. The chronic and acute ambulatory trends are stored for predetermined time periods. The physician is provided means for accessing the data to assess and improve the prescribed CRT. The CRM device has a finite memory for storing data. In various embodiments, the CRM device periodically uploads data to an external device, such as an APM device, which provides the ability to maintain a longer term trend or more detailed trend.

In various embodiments, the data is stored upon detecting a triggering condition indicating CRT degradation. The triggering conditions that initiate such data storage may relate, for example, to situations in which there is a reduced frequency of pacing in one or more of the device's pacing channels. The stored data may take the form of electrograms recorded from one or more sensing channels or data derived therefrom such as intervals between detected events.

In various embodiments of the present subject matter, the CRM device is configured to deliver CRT and is further configured to store meaningful data useful in evaluating a status of the CRM device and the efficacy of the CRT. What follows is a description of the method as well as of the hardware components and operating modes of a device in which the method may be implemented.

1. Hardware Platform for CRM Device

In the embodiment to be described, the invention is implemented with a control unit made up of a microprocessor executing programmed instructions in memory. However, certain functions of a CRM device could be controlled by custom logic circuitry either in addition to or instead of a programmed microprocessor. The term “controller” as used herein encompasses custom circuitry (i.e., dedicated hardware) and/or a microprocessor executing programmed instructions contained in a processor-readable storage medium along with associated circuit elements.

Pacemakers and other types of implantable CRM devices are typically implanted subcutaneously or submuscularly in a patient's chest and have leads threaded intravenously into the heart to connect the device to electrodes used for sensing and pacing. Leads may also be positioned on the epicardium by various means. A programmable electronic controller causes the pacing pulses to be output in response to lapsed time intervals and sensed electrical activity (i.e., intrinsic heart beats not as a result of a pacing pulse). Pacemakers sense intrinsic cardiac electrical activity by means of internal electrodes disposed near the chamber to be sensed. A depolarization wave associated with an intrinsic contraction of the atria or ventricles that is detected by the pacemaker and exceeds a specified threshold is referred to as an atrial sense or ventricular sense, respectively. In order to cause such a contraction in the absence of an intrinsic beat, a pacing pulse (either an atrial pace or a ventricular pace) with energy above a certain pacing threshold is delivered to a heart chamber.

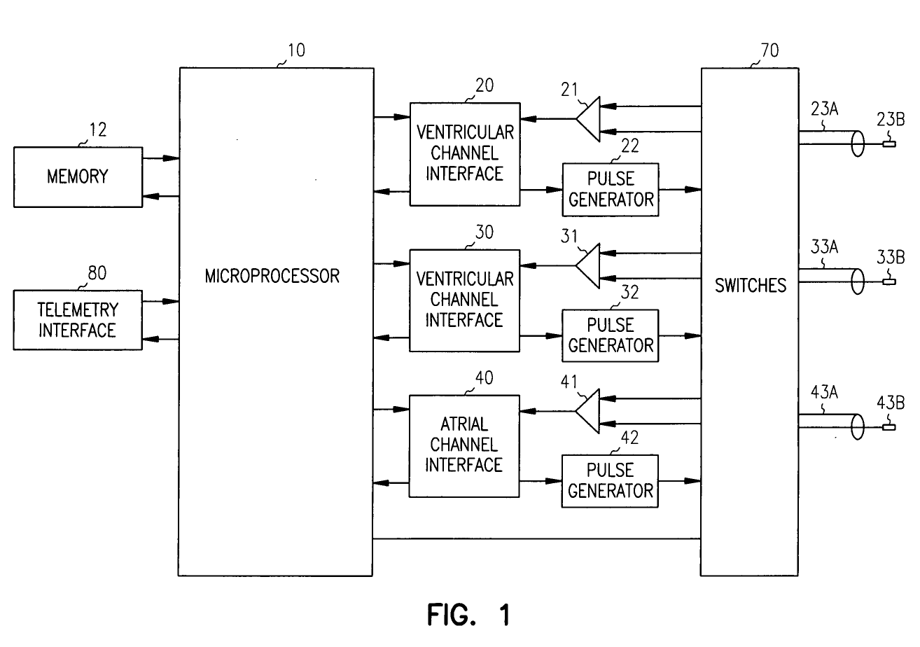

FIG. 1 illustrates a block diagram of a multi-site pacemaker configurable to deliver CRT, according to various embodiments of the present subject matter. The illustrated pacemaker includes three sensing/pacing channels. As the term is used herein, a “pacemaker” should be taken to mean any CRM device, such as an implantable cardioverter/defibrillator, with a pacing functionality. The control unit of the pacemaker is made up of a microprocessor 10 communicating with a memory 12 via a bidirectional data bus, where the memory 12 typically comprises a ROM (read-only memory) and a RAM (random-access memory) for program and data storage. The control unit could be implemented by other types of logic circuitry (e.g., discrete components or programmable logic arrays) using a state machine type of design, but a microprocessor-based system is preferable. The control unit is capable of operating the pacemaker in a number of programmed modes where a programmed mode defines how pacing pulses are output in response to sensed events and expiration of time intervals. A telemetry interface 80 is also provided for communicating with an external programmer.

In various embodiments the multiple sensing/pacing channels are configured to deliver biventricular pacing. In various embodiments the multiple sensing/pacing channels are configured to deliver biatrial pacing. In various embodiments the multiple sensing/pacing channels are configured to deliver multi-site pacing of a single chamber. The illustrated pacemaker includes one atrial and two ventricular sensing/pacing channels for delivering biventricular pacing. The atrial sensing/pacing channel in FIG. 1 comprises ring electrode 43a, tip electrode 43b, sense amplifier 41, pulse generator 42, and an atrial channel interface 40 which communicates bidirectionally with a port of microprocessor 10. The device also has two ventricular sensing/pacing channels that similarly include ring electrodes 23a and 33a, tip electrodes 23b and 33b, sense amplifiers 21 and 31, pulse generators 22 and 32, and ventricular channel interfaces 20 and 30. Incorporated into each sensing/pacing channel is thus a pacing channel made up of the pulse generator connected to the electrode and a sensing channel made up of the sense amplifier connected to the electrode. The channel interfaces include analog-to-digital converters for digitizing sensing signal inputs from the sensing amplifiers, registers that can be written to for adjusting the gain and threshold values of the sensing amplifiers, and registers for controlling the output of pacing pulses and/or changing the pacing pulse amplitude. For each channel illustrated in FIG. 1, the same electrode pair is used for both sensing and pacing. In this embodiment, bipolar leads that include two electrodes are used for outputting a pacing pulse and/or sensing intrinsic activity. Other embodiments may employ a single electrode for sensing and pacing in each channel, known as a unipolar lead. A switching network 70, such as a metal oxide semiconductor (MOS) switching network, controlled by the microprocessor is used to switch the electrodes from the input of a sense amplifier to the output of a pulse generator.

The controller 10 controls the overall operation of the device in accordance with programmed instructions stored in memory. The controller 10 interprets sense signals from the sensing channels and controls the delivery of paces in accordance with a pacing mode. The sensing circuitry of the pacemaker generates atrial and ventricular senses when voltages sensed by the electrodes of a particular channel exceed a specified threshold. Pacing algorithms employ such senses to trigger or inhibit pacing. The sense signals from each channel can also be recorded in memory for a specified period of time to constitute an electrogram that can later be transmitted via the telemetry link to an external programmer. An electrogram is analogous to a surface ECG and provides a temporal record of cardiac depolarization and repolarization that occurs during either intrinsic or paced beats. In various embodiments, the recording of an electrogram may be triggered by the detection of certain events or conditions such as the onset of a tachyarrhythmia in order to provide diagnostic information to a clinician. As described below, electrograms can also provide useful information in evaluating the functioning of the device in providing cardiac resynchronization therapy.

2. Bradycardia Pacing Modes

Bradycardia pacing modes refer to pacing algorithms used to pace the atria and/or ventricles when the intrinsic atrial and/or ventricular rate is inadequate due to, for example, AV conduction blocks or sinus node dysfunction. Such modes may either be single-chamber pacing, where either an atrium or a ventricle is paced, or dual-chamber pacing in which both an atrium and a ventricle are paced. The modes are generally designated by a letter code of three positions where each letter in the code refers to a specific function of the pacemaker. The first letter refers to which heart chambers are paced and which may be an A (for atrium), a V (for ventricle), D (for both chambers), or O (for none). The second letter refers to which chambers are sensed by the pacemaker's sensing channels and uses the same letter designations as used for pacing. The third letter refers to the pacemaker's response to a sensed P wave from the atrium or an R wave from the ventricle and may be an I (for inhibited), T (for triggered), D (for dual in which both triggering and inhibition are used), and O (for no response). Modern pacemakers are typically programmable so that they can operate in any mode which the physical configuration of the device will allow. Additional sensing of physiological data allows some pacemakers to change the rate at which they pace the heart in accordance with some parameter correlated to metabolic demand. Such pacemakers are called rate-adaptive pacemakers and are designated by a fourth letter added to the three-letter code, R.

Pacemakers can enforce a minimum heart rate either asynchronously or synchronously. In asynchronous pacing, the heart is paced at a fixed rate irrespective of intrinsic cardiac activity. There is thus a risk with asynchronous pacing that a pacing pulse will be delivered coincident with an intrinsic beat and during the heart's vulnerable period which may cause fibrillation. Most pacemakers for treating bradycardia today are therefore programmed to operate synchronously in a so-called demand mode where sensed cardiac events occurring within a defined interval either trigger or inhibit a pacing pulse. Inhibited demand pacing modes utilize escape intervals to control pacing in accordance with sensed intrinsic activity. In an inhibited demand mode, a pacing pulse is delivered to a heart chamber during a cardiac cycle only after expiration of a defined escape interval during which no intrinsic beat by the chamber is detected. If an intrinsic beat occurs during this interval, the heart is thus allowed to “escape” from pacing by the pacemaker. Such an escape interval can be defined for each paced chamber. For example, a ventricular escape interval can be defined between ventricular events so as to be restarted with each ventricular sense or pace. The inverse of this escape interval is the minimum rate at which the pacemaker will allow the ventricles to beat, sometimes referred to as the lower rate limit (LRL).

In atrial tracking pacemakers (i.e., VDD or DDD mode), another ventricular escape interval is defined between atrial and ventricular events, referred to as the atrio-ventricular interval (AVI). The AV interval is triggered by an atrial sense or pace and stopped by a ventricular sense or pace. A ventricular pace is delivered upon expiration of the AV interval if no ventricular sense occurs before. Atrial-tracking ventricular pacing attempts to maintain the atrio-ventricular synchrony occurring with physiological beats whereby atrial contractions augment diastolic filling of the ventricles. If a patient has a physiologically normal atrial rhythm, atrial-tracking pacing also allows the ventricular pacing rate to be responsive to the metabolic needs of the body. However, such tracking may not be appropriate where the patent has atrial arrhythmia.

A pacemaker can also be configured to pace the atria on an inhibited demand basis. An atrial escape interval is then defined as the maximum time interval in which an atrial sense must be detected after a ventricular sense or pace before an atrial pace will be delivered. When atrial inhibited demand pacing is combined with atrial-triggered ventricular demand pacing (i.e., DDD mode), the lower rate limit interval is then the sum of the atrial escape interval and the atrio-ventricular interval.

Another type of synchronous pacing is atrial-triggered or ventricular-triggered pacing. In this mode, an atrium or ventricle is paced immediately after an intrinsic beat is detected in the respective chamber. Triggered pacing of a heart chamber is normally combined with inhibited demand pacing so that a pace is also delivered upon expiration of an escape interval in which no intrinsic beat occurs. Such triggered pacing may be employed as a safer alternative to asynchronous pacing when, due to far-field sensing of electromagnetic interference from external sources or skeletal muscle, false inhibition of pacing pulses is a problem. If a sense in the chamber's sensing channel is an actual depolarization and not a far-field sense, the triggered pace is delivered during the chamber's physiological refractory period and is of no consequence.

Finally, rate-adaptive algorithms may be used in conjunction with bradycardia pacing modes. Rate-adaptive pacemakers modulate the ventricular and/or atrial escape intervals based upon measurements corresponding to physical activity. Such pacemakers are applicable to situations in which atrial tracking modes cannot be used. In a rate-adaptive pacemaker operating in a ventricular pacing mode, for example, the LRL is adjusted in accordance with exertion level measurements such as from an accelerometer or minute ventilation sensor in order for the heart rate to more nearly match metabolic demand. The adjusted LRL is then termed the sensor-indicated rate.

3. Cardiac Resynchronization Therapy (CRT)

CRT applies pacing stimulation applied to one or more heart chambers in a manner that restores or maintains synchronized contractions of the atria and/or ventricles and thereby improves pumping efficiency. Although resynchronization pacing may be delivered to only one heart chamber or multiple sites of a single chamber, it most often involves pacing both ventricles in accordance with a biventricular resynchronization pacing mode as described below. Ventricular resynchronization pacing is useful in treating heart failure because, although not directly ionotropic, resynchronization results in a more coordinated contraction of the ventricles with improved pumping efficiency and increased cardiac output.

One way to deliver resynchronization therapy is to pace a site with a synchronous bradycardia pacing mode and then deliver one or more resynchronization paces to one or more additional pacing sites in a defined time relation to one or more selected sensing or pacing events that either reset escape intervals or trigger paces in the bradycardia pacing mode. The site paced with the bradycardia mode may be referred to as a rate site or rate chamber, while the site paced with resynchronization paces may be referred to as a synchronized site or synchronized chamber. One such resynchronization pacing mode may be termed offset resynchronization pacing. In this mode, a first site is paced with a bradycardia mode, and a second site receives a resynchronization pace at an offset interval with respect to the pace delivered to the first site. The offset interval may be zero in order to pace both sites simultaneously, positive in order to pace the first site after the second, or negative to pace the first site before the second. For example, in biventricular resynchronization pacing, one ventricle is paced with a bradycardia mode while the contralateral ventricle receives resynchronization paces at the specified biventricular offset interval. The offset interval would normally be individually specified to optimize cardiac output in a particular patient. Ventricular resynchronization can also be achieved in certain patients by pacing at a single unconventional site, such as the left ventricle instead of the right ventricle. In such a mode, right ventricular senses may be used to trigger left ventricular paces or used to define an escape interval that upon expiration causes delivery of a left ventricular pace (i.e., the right ventricle is a rate chamber and the left ventricle is a synchronized ventricle).

Another synchronized mode is triggered synchronized pacing. In one type of triggered synchronized pacing, the synchronized chamber is paced after a specified trigger interval following a sense in the rate chamber, while in another type the rate chamber is paced after a specified trigger interval following a sense in the synchronized chamber. The two types may also be employed simultaneously. For example, with a trigger interval of zero, pacing of one chamber is triggered to occur in the shortest time possible after a sense in the other chamber in order to produce a coordinated contraction. The shortest possible time for the triggered pace is limited by a sense-to-pace latency period dictated by the hardware. This mode of pacing may be desirable when the intra-chamber conduction time is long enough that the pacemaker is able to reliably insert a pace before depolarization from one chamber reaches the other. Triggered synchronized pacing can also be combined with offset synchronized pacing such that both chambers are paced with the specified offset interval if no intrinsic activity is sensed in the rate chamber and a pace to the rate chamber is not otherwise delivered as a result of a triggering event. A specific example of this mode is ventricular triggered synchronized pacing where the rate and synchronized chambers are the right and left ventricles, respectively, and a sense in the right ventricle triggers a pace to the left ventricle and/or a sense in the left ventricle triggers a pace to the right ventricle.

As with other synchronized pacing modes, the rate chamber in a triggered synchronized pacing mode can be paced with one or more synchronous bradycardia pacing modes. If the rate chamber is controlled by a triggered bradycardia mode, a sense in the rate chamber sensing channel, in addition to triggering a pace to the synchronized chamber, also triggers an immediate rate chamber pace and resets any rate chamber escape interval. The advantage of this modal combination is that the sensed event in the rate chamber sensing channel might actually be a far-field sense from the synchronized chamber, in which case the rate chamber pace should not be inhibited. In a specific example, the right and left ventricles are the rate and synchronized chambers, respectively, and a sense in the right ventricle triggers a pace to the left ventricle. If right ventricular triggered pacing is also employed as a bradycardia mode, both ventricles are paced after a right ventricular sense has been received to allow for the possibility that the right ventricular sense was actually a far-field sense of left ventricular depolarization in the right ventricular channel. If the right ventricular sense were actually from the right ventricle, the right ventricular pace would occur during the right ventricle's physiological refractory period and cause no harm.

In the synchronized modes described above, the rate chamber is synchronously paced with a mode based upon detected intrinsic activity in the rate chamber, thus protecting the rate chamber against paces being delivered during the vulnerable period. In order to provide similar protection to a synchronized chamber or synchronized pacing site, a synchronized chamber protection period (SCPP) may be provided. In the case of multi-site synchronized pacing, a similar synchronized site protection period may be provided for each synchronized site. The SCPP is a programmed interval which is initiated by sense or pace occurring in the synchronized chamber during which paces to the synchronized chamber are inhibited. For example, if the right ventricle is the rate chamber and the left ventricle is the synchronized chamber, a left ventricular protection period LVPP is triggered by a left ventricular sense which inhibits a left ventricular pace which would otherwise occur before the escape interval expires. The SCPP may be adjusted dynamically as a function of heart rate and may be different depending upon whether it was initiated by a sense or a pace. The SCPP thus provides a means to inhibit pacing of the synchronized chamber when a pace might be delivered during the vulnerable period or when it might compromise pumping efficiency by pacing the chamber too close to an intrinsic beat.

4. Triggered Storage of Diagnostic Data

Various embodiments of the present subject matter store diagnostic data based on a triggered event. Thus, in these embodiments, the device does not continuously record data, but rather records data in response to a triggering event. In the description that follows, a device such as that depicted in FIG. 1 is assumed to be configured to deliver cardiac resynchronization therapy (CRT) in a biventricular pacing mode with the right ventricle serving as the rate chamber. Other embodiments may employ the left ventricle as the rate chamber. When such a device is operating in a patient, only cardiac cycles in which both ventricles are paced have the benefit of being resynchronized. It is therefore desirable for pacing to be frequent, and parameter settings or conditions that allow more natural beats to occur reduce the effectiveness of the therapy.

A number of factors can result in a loss of or reduction of pacing to either or both ventricles in a biventricular resynchronization pacing mode. For example, in a VVI or VVIR mode, ventricular paces will be inhibited by intrinsic activity, and less frequent pacing will result if the LRL is set too low or, in the case of rate-adaptive pacing, if the exertion level sensor does not adequately increase the LRL. Similarly, in an atrial tracking mode, if the patient's intrinsic PR interval is too short as compared with the programmed AV escape interval, intrinsic ventricular beats will occur more frequently. Maximum limits on the pacing rate (e.g., limits on the sensor-indicated rate or the atrial tracking rate) that are too low can also result in more frequent intrinsic beats than are desirable, as can incorrect parameter settings of pacing modes in which the escape intervals are dynamically adjusted in order to suppress intrinsic beats (i.e., ventricular rate regularization or other types of overdrive pacing).

Underpacing may also be the result of oversensing in a sensing channel that either restarts escape intervals or triggers protective periods. Excessive noise or inadequate refractory periods in the rate channel would result in underpacing of both ventricles, while such noise or under-refractoriness in the synchronized channel would cause underpacing of only the synchronized chamber. For example, in RV-based biventricular pacing, noise in the LV channel or an LVPP (left ventricular protection period) that is too long would cause underpacing of only the left ventricle. Underpacing of only the synchronized chamber can also occur if the offset interval becomes too short due to, for example, changes in the intrinsic RV-LV conduction interval. Other factors, such as insufficient pacing energy which can come about due to equipment failure or changes in the myocardial substrate, may reduce the effectiveness of pacing without necessarily affecting the pacing frequency.

When a patient undergoing resynchronization therapy is evaluated during follow-up, clinical symptoms or other indicia may indicate that the therapy has become less effective and that some adjustments should be made to improve the efficacy of the CRT. It may be difficult to ascertain, however, exactly what parameters should be changed in order to re-optimize the therapy. Although the operation of the implanted device may be monitored with an external programmer, some of the problems listed above may only occur intermittently or under special circumstances. It would therefore be useful for the physician to have diagnostic data that is recorded by the device during the time at which an instance of compromised resynchronization therapy occurred.

The present invention provides a method for operating a cardiac rhythm management device in which CRT-related data is recorded for use to evaluate a status of the CRT. Various embodiments trigger the recording of data (e.g. sensed data, program parameters, time, etc.) upon detection of a condition that indicates some degradation in CRT. Such a triggering condition may be based upon a presently detected parameter or a parameter derived from data collected over a specified time period. For example, the device may maintain running totals of the number of paced and unpaced cycles so that data storage is triggered when underpacing is detected such as: if the percent of paced cycles over a certain period of time in either or both ventricles drops below a specified threshold or drops below a specified threshold within a particular rate range, or if the number of consecutive intrinsic beats exceeds a specified threshold. The device may also keep track of other parameters that may be used to trigger recording of data such as if the number of times a pace has been inhibited by the synchronized-chamber protective period within a specified time interval exceeds a specified limit value, or if the number of triggered paces over specified time interval exceeds a specified limit value. The device may also periodically measure the intrinsic PR interval by detecting the time interval between atrial and ventricular senses during unpaced beats so that data storage is triggered when the measured PR interval has deviated a defined percentage from the intrinsic PR interval measured during the last optimization. Another possible triggering condition is whenever the prescribed (i.e. programmed) cardiac synchronization therapy is consistently not delivered. For example the programmed pacing site(s), AV delay, and synchronization chamber offset can be defined as the pertinent programmed cardiac synchronization therapy parameters. Then, whenever it is detected that therapy is not delivered according to these parameters and at a predetermined consistency, data storage is triggered.

Since the device may have multiple triggering conditions, another beneficial capability would be to indicate which triggering condition caused a particular storage event. Additional statistical information regarding the condition may therefore be stored, such as the actual percentage of paced ventricular beats or average PR interval for a specified time interval preceding the event. Also, additional data regarding the condition of the patient may be useful in diagnosing the problem associated with the data storage. Therefore the device may also store items such as atrial rate, ventricular rate, activity level, respiration rate, autonomic balance, date, or time. In addition to the data stored when a triggering condition is met, the system could also continuously store information regarding the triggering parameters. In one embodiment, for example, the device could continuously store the percentage of time resynchronization therapy is being delivered. This data could then be displayed as a percentage trend over time.

In many instances, it is likely that the conditions that trigger storage of data could occur many times in a relatively short period of time due to essentially the same problem. In such cases, storage of what would amount to multiple data records that relate to that problem would waste memory and provide little diagnostic value over a single storage event. To more effectively utilize the limited memory in the implanted device and to minimize data presentation to the user, the device may limit the number of storage events that are due to a particular triggering condition within a specified period of time.

It may be important to inhibit triggering of data storage while certain pathological conditions exist. For example, during an episode of ventricular tachycardia or ventricular fibrillation, delivery of cardiac resynchronization therapy is normally inhibited. Therefore, although the conditions for triggering data storage may be otherwise met, storing data for the purposes of diagnosing lack of cardiac resynchronization therapy would be inappropriate in this situation. Other examples of conditions where data storage may be withheld or the conditions for triggering data storage may be modified include atrial arrhythmias, device malfunction and temporary programming used for diagnostic purposes.

When a triggering condition is detected, the device begins recording data from the atrial and/or ventricular sensing channels for a specified storage time. In one embodiment, the recorded data are electrograms represented by the digitized and stored voltage values received by the sensing channels. Since it is often useful for the physician to understand the situation immediately prior to the time when the triggering condition is detected, electrograms reflecting data collected just prior to detection of the triggering condition could be stored as well as those reflecting subsequently collected data. This may be implemented by the device continuously storing data in a first-in-first-out queue. The data in the queue can then be stored in a more permanent storage location along with subsequently collected data when a triggering condition is detected.

Storing electrograms requires a considerable amount of memory, typically 150-200 bytes per cardiac cycle. As an alternative, therefore, markers representing sensed and paced events along with time intervals between the events may be stored instead. Another alternative for reducing the amount of memory required is to retain less information on older events. In one embodiment electrograms, markers and intervals would be stored for the most recent events where detailed information is most useful and only markers and intervals would be stored for older events where less information may be required.

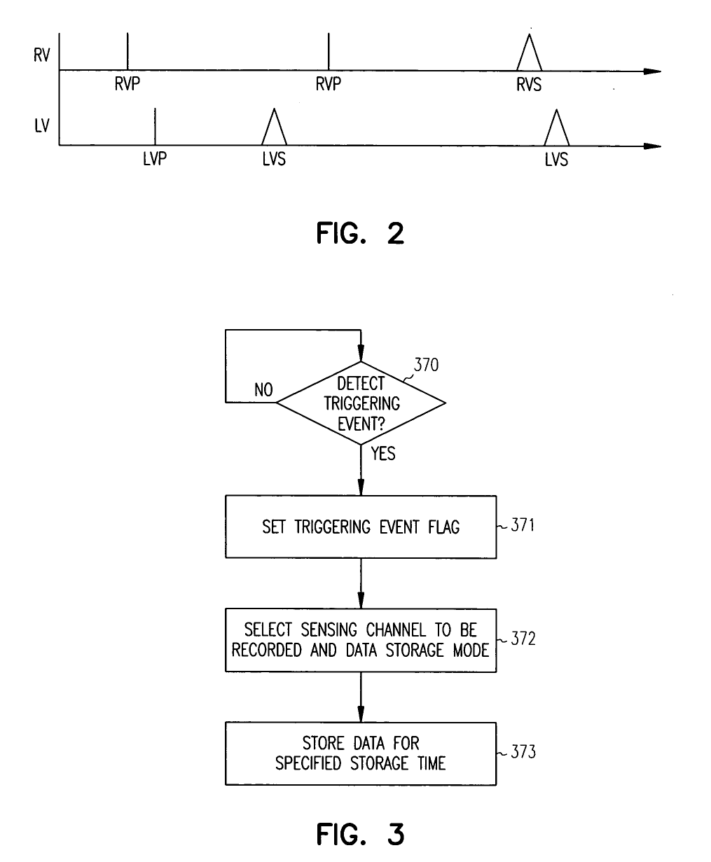

FIG. 2 illustrates a display of marker/interval data, according to various embodiments of the present subject matter. An external programmer or advanced patient management (APM) device display these markers in embodiments of the present subject matter. Timelines are displayed representing electrogram data for the right and left ventricular sensing/pacing channels, labeled RV and LV, respectively. Underneath the timelines are two lines of markers output by the display for both ventricular channels. Each marker in the top line represents right ventricular events, and each marker in the bottom line represents left ventricular events. Associated with each marker is an intraventricular interval which indicates the time interval between the event represented by the marker and another ventricular event. In this example, the interval displayed with each right and left ventricular marker is the interval from the previous right ventricular event. Storing markers and intervals requires considerably less memory, on the order of 8-10 bytes per cardiac cycle.

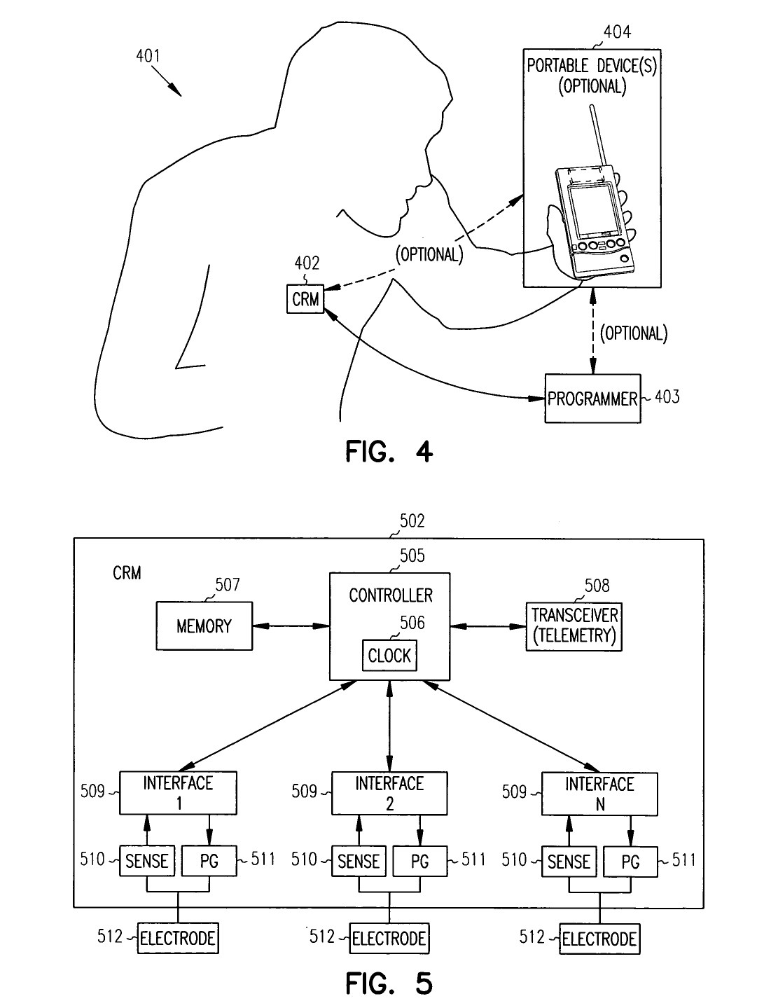

Various embodiments of the CRM device are capable of transmitting the stored data to an external device other than a programmer. Examples of such a device include a personal digital assistant (PDA), a local computer, and networked computer such as may be implemented in an advanced patient management (APM) system. FIG. 4 illustrates the use of a PDA. Since data can be periodically uploaded from the CRM device to the external device which has more memory and/or removable memory, the external device alleviates data storage issues.

Many CRT problems can be diagnosed with only markers and intervals. Electrograms, however, may be beneficial in some cases and essential in others. Also, many CRT problems can be diagnosed with electrograms from only two channels, but the particular two channels that are required depends upon the specific problem. For example, loss of pacing in both ventricular channels due to the intrinsic PR interval being shorter than the programmed atrio-ventricular interval can be diagnosed with data from the atrial and right ventricular channels. Loss of left ventricular pacing due to far-field sensing of right ventricular depolarizations in the left ventricular channel, on the other hand, is best diagnosed with data from the left and right ventricular channels. If memory constraints prevent recording data from all three sensing channels, the particular channels from which diagnostic data is to be stored may be selected in accordance with the particular triggering event that is detected. Whether an electrogram or marker/interval data is stored may also be made to depend upon the particular triggering event.

FIG. 3 is a flowchart illustrating a triggered data storage method, according to various embodiments of the present subject matter. Various embodiments implement the method through programming of the controller. A triggering event is looked for at 370, and, if one is detected, a flag indicating the event is set at 371. The sensing channels from which diagnostic data is to be recorded and the storage mode to be used, either electrograms or marker/interval data, are then selected at 372 in accordance with the particular triggering event detected at 370. Diagnostic data is then recorded for a defined storage time at 373.

5. Trended CRT

According to various embodiments of the present subject matter, CRT-related data indicative of the efficacy of CRT (e.g., data corresponding to the successful delivery of prescribed CRT therapy) is measured and stored to maintain a history of the CRT therapy, at least on a periodic basis. Various embodiments use a time-based approach and various embodiments use an event-based approach to quantify successful delivery of CRT therapy. Those of ordinary skill in the art will understand, upon reading and comprehending this disclosure, that the efficacy of the CRT can be determined based on recording data related to successful CRT events, data related to unsuccessful CRT events, and data related to both successful and unsuccessful CRT events. Various embodiments are responsive to a triggering event to record a trend for a period of time.

An example of a time-based approach involves measuring the amount of time (e.g. time contribution of each cardiac cycle) in which CRT is successfully delivered and the total time. An example of an event-based approach involves measuring the number of events in which CRT therapy is successfully delivered and the total number of events. The ratio of these time-based or event-based measurements can be used to quantify the successful delivery and efficacy of a prescribed CRT. Examples of such realized CRT data include CRT delivery, % RV pace, % LV pace, % AT, % capture, % above MTR/MSR/MPR, TT (total time in tracking mode during period), TN (total time in non-tracking mode during period), TTCRT (total time CRT is successfully delivered in tracking mode), and TNCRT (total time CRT is successfully delivered in non-tracking mode). These examples of realized CRT data can be provided as an absolute value, such as time or event, or as a percentage.

Since the operating mode of the CRM device is likely to affect the level of successful delivery of prescribed CRT, various embodiments of the present subject matter maintain separate data storage files classified according to an operating mode of the CRM device. For example, since the likelihood of successful CRT delivery may be greater when the CRM device is operating in tracking modes versus non-tracking modes, various embodiments maintain separate measurements for atrial tracking modes and non-tracking modes. The stored data is used to provide a means for assessing a status of a prescribed CRT. Various embodiments organize and illustrate the data as graphical trends. Various embodiments organize and illustrate the data in tables. Various embodiments implement algorithms to process the data to otherwise notify, or alert a physician of the efficacy of the prescribed CRT.

6. Example of Trended Measurement of CRT Delivery

An example is provided herein to illustrate trended measurement of CRT delivery. This example is not intended to identify all potential implementations, and thus should not be read to limit the present subject matter. This example distinguishes when a CRM device is operating in an atrial tracking mode and when the CRM device is operating in a non-atrial tracking mode. This example also uses a time-based approach to quantify successful CRT delivery with respect to total attempted CRT delivery to determine the status of the CRM device in delivery effective CRT. Five cases are illustrated in Table 1.

TABLE 1

| | 1 | 2 | 3 | 4 | 5 |
|---|---|---|---|---|---|
| TT₂₄ | 24 Hr | 0 Hr | 23 Hr | 1 Hr | 20 Hr |
| TN₂₄ | 0 Hr | 24 Hr | 1 Hr | 23 Hr | 4 Hr |
| TTCRT₂₄ | 0 Hr | 0 Hr | 1 Hr | 1 Hr | 19 Hr |
| TNCRT₂₄ | 0 Hr | 0 Hr | 1 Hr | 1 Hr | 3 Hr |
| LastATrackModeCRTPercent | 0% | NA | 4% | 100% | 95% |
| LastNonATrackModeCRTPercent | NA | 0% | 100% | 4% | 75% |

In Table 1, TT₂₄ represents the total time that the CRM device is operating in a tracking mode over a 24-hour period, TTCRT₂₄ represents the total time CRT therapy is successfully delivered while the CRT is operating in a tracking mode over a 24-hour period, TN₂₄ represents the total time the CRM device is operating in a non-tracking mode over a 24-hour period, and TNCRT₂₄ represents the total time CRT therapy is successfully delivered while the CRM device is operating in a non-tracking mode over a 24-hour period. A 24-hour period is illustrated as an example. Other periods, such as 1-hour periods, can be used to facilitate identifying dependences related to activities or time of day. LastATrackModeCRTPercent and LastNonATrackModeCRTPercent are intended to provide a measure of the successful delivery of CRT while the CRM device is operating in tracking and non-tracking modes, respectively. These two parameters can range from 0% (no successful CRT delivery) to 100% (all successful CRT delivery). The appropriate output is flagged as not applicable (NA) if the CRM device did not operate in both tracking and non-tracking modes. Rather than quantify the efficacy of CRT in terms of percentage, absolute quantities can be used. In a time-based example, LastATrackModeCRT and LastNonATrackModeCRT can be expressed in terms of a time quantity (e.g., hours or minutes) of a predetermined time frame (e.g. day). In an event-based example, LastATrackModeCRT and LastNonATrackModeCRT can be expressed as a number of events for a predetermined time frame (e.g. day).

In the first case illustrated in Table 1, the CRM device is operated only in tracking modes. CRT was successfully delivered 0% of the time while the CRM device was operating in a tracking mode. A physician or clinician may likely determine that it is warranted to modify parameters to improve CRT delivery success in tracking modes.

In the second case illustrated in Table 1, the CRM device is operated only in non-tracking modes. CRT was successfully delivered 0% of the time while the PG was operating in a non-tracing mode. A physician or clinician may likely determine that it is warranted to modify parameters to improve CRT delivery success in non-tracking modes.

In the third case illustrated in Table 1, the CRM device is operated in both tracking and non-tracking modes. CRT was successfully delivered 4% of the time while the CRM device was operating in a tracking mode and 100% of the time while the CRM device was operating in a non-tracking mode. A physician or clinician may likely determine that it is warranted to modify parameters to improve CRT delivery success in tracking modes.

In the fourth case illustrated in Table 1, the CRM device is operated in both tracking and non-tracking modes. CRT was successfully delivered 100% of the while the CRM device was operating in a tracking mode and 4% of the time while the CRM device was operating in a non-tracking mode. A physician or clinician may likely determine that it is warranted to modify parameters to improve CRT delivery success in non-tracking modes.

In the fifth case illustrated in Table 1, the CRM device is operated in both tracking and non-tracking modes. CRT was successfully delivered 95% of the time while the CRM device was operating in a tracking mode and 75% of the time while the CRM device was operating in a non-tracking mode. A physician or clinician may likely determine that it is necessary to modify parameters to improve CRT delivery.

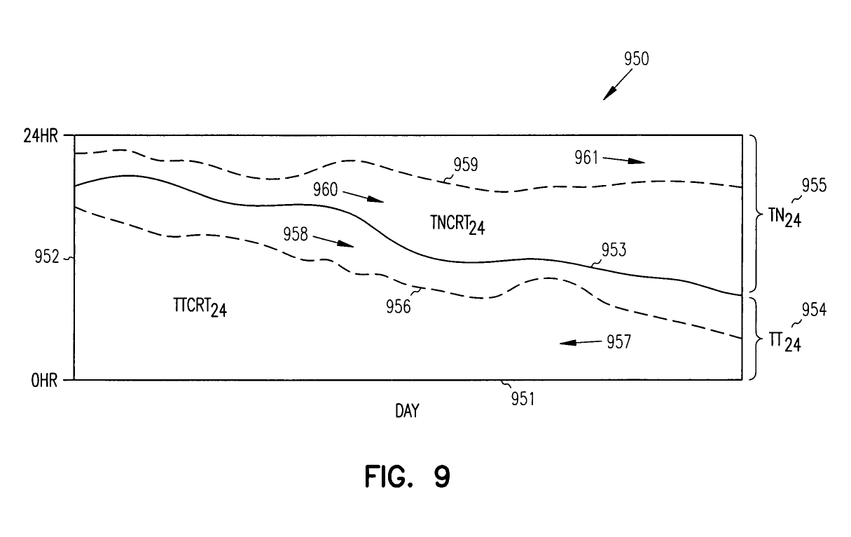

An example of a trend is illustrated in and discussed below with respect to FIG. 9. Various embodiments trend over a period of time such as a day, and as such, are able to provide an indication of a dependence on the time of day. Thus, a clinician is able to correlate patient activities with changes in the CRT delivery success. For example, with an appropriately small measurement period, sleep or physical activity is able to be correlated with changes in CRT delivery success, thus providing the clinician with the ability to improve the therapy.

7. Other Trended CRT-Related Parameters

A number of CRT-related parameters can be trended, according to various embodiments of the present subject matter. The trended parameters include programmed parameters corresponding to the prescribed therapy, and the realized CRT data.

Examples of programmed parameters corresponding to prescribed therapy include AV Delay, Pacing Mode, Ventricular Pacing Chamber {RV, LV, BiV }, MTR, LRL, LV Offset, Sensed AV Offset, VRR, BiV Trigger, LVPP, Refractory Windows, etc. These programmable CRT parameters, along with others, are capable of affecting the efficacy of CRT. Various embodiments of the present subject matter record these parameters along with the corresponding success rate of the CRT. This information is useful for a physician or clinician in assessing and improving the efficacy of the CRT.

Examples of realized CRT data include a time in tracking mode parameter (TT), a time in non-tracking mode parameter (TN), a total time CRT delivery is successful in a tracking mode (TTCRT) and a total time CRT delivery is successful in a non-tracking mode (TNCRT), such as is illustrated above. Other examples of realized CRT data include the percent RV pace, the percent LV pace, the percent at the prescribed offset pace, the percent capture, the percent above MTR, the percent above MSR, and the percent above MPR. These and other realized CRT data are capable of being trended according to various embodiments of the present subject matter.

8. Parameter Trending Examples

Various trending examples are provided herein. These examples are not intended to be an exclusive listing of the trending parameters.

Various trending embodiments trend N samples per unit time (such as 1 sample per day, for example). Various trending embodiments trend N samples per unit time until a predetermined change occurs, and then M samples per unit time are trended. An example of a predetermined change is a 10% drop in paced cycles in the preceding day. Various trending embodiments trend N samples per unit time until a predetermined threshold is reached, and then M samples per unit time are trended. An example of a predetermined threshold is 70% pacing. Various trending embodiments trend N samples per unit time until a predetermined event occurs, and then M samples per unit time are trended. An example of a predetermined event is the detection of edema. Various trending embodiments trend M samples per unit time after being triggered by a predetermined change, after reaching a predetermined threshold, or after a predetermined event. Various trending embodiments trend a different parameter after being triggered by a predetermined change, after reaching a predetermined threshold, or after a predetermined event.

For example, various embodiments of the present subject matter initiate an action if the sum of TTCRT and/or TNCRT falls below an established threshold. Various embodiments initiate an action if TTCRT/TT and/or TNCRT/TN falls below an established threshold. Various embodiments initiate an action if TT falls below an established threshold. According to some embodiments, the initiated action includes an alert or notification to the clinician, patient, and the like. According to some embodiments, the initiated action includes an automatic action such as triggering a patient-triggered monitor, or changing a prescribed CRT.

9. Systems

A CRM device has been previously illustrated and discussed with respect to FIG. 1. The CRM device illustrated in FIG. 1 includes two ventricular channel interfaces and an atrial channel interface. According to various embodiments, the trended measurement of CRT-related data is implemented in other CRM device configurations and in various systems that include an implantable CRM device.

FIG. 4 illustrates a system 401 that includes an implantable CRM device 402, a programmer 403, and an optional portable advance patient management (APM) device 404, according to various embodiments of the present subject matter. A CRM device 402, such as an implantable pacemaker with or without defibrillation capabilities, is implanted in a patient. The illustrated CRM device 402 includes circuitry and software to deliver CRT. The illustrated system 401 also includes a programmer 403, such as is used by a physician or clinician to program the implantable CRM device 402. Both the CRM device 402 and the programmer 403 include communication circuitry that enable the CRM device and the programmer to communicate with each other. For example, various embodiments include telemetry circuitry or other circuitry, referred to herein as transceivers, suitable for transmitting and receiving wireless communication signals.

CRT-related data corresponding to the status of the CRT is measured by the CRM device. This measured data is referred to herein as raw CRT-related data. CRT-related data trends are capable of being displayed on a monitor of the programmer. Various embodiments process the raw CRT-related data in the CRM device store processed data in the CRM device. Various embodiments store the raw CRT-related data in the CRM device and process this data in the programmer. Various embodiments process the raw CRT-related data partially in the CRM device and partially in the programmer.

The CRM device 402 has limited memory. Thus, the memory may limit the amount of recorded CRT-related data, depending on the number of recorded data parameters and/or the length of time for recording the CRT-related data. Therefore, various embodiments use a portable device, such as a personal digital assistant (PDA), a laptop computer and the like with more memory and/or removable memory, to store the data to provide the desired trends. Additionally, the portable devices can include stand alone devices and/or networked devices. Examples of such portable devices have been implemented in patient wellness monitors within advanced patient management (APM) systems, such as has been described in U.S. patent application Ser. No. 10/323859, which is herein incorporated by reference in its entirety. A benefit of APM systems is the ability to acquire, and thus trend, realized data, prescribed data, mode, time, and other data that may affect the delivery of CRT.

Thus, a basic implementation of a system to provide trends of measured CRT-related data representative of the status of CRT includes a CRM device and a programmer. A physician or clinician is capable of assessing the chronic, ambulatory status of the CRT using the programmer, such as using displays of trends on a monitor of the programmer. Based on this assessment, the physician or clinician is able to intelligently adjust the programming of the CRM device to improve CRT. However, the present subject matter is not limited to this basic implementation, as generally illustrated by the incorporation of the optional portable device in the system illustrated in FIG. 4.

FIG. 5 illustrates an implantable CRM device such as can be used in the system of FIG. 4, according to various embodiments of the present subject matter. The illustrated CRM device 502 includes a processor or controller 505 with a clock 506. The clock 506 is used to provide time information (e.g. identify the time and day, etc.) when the raw CRT-related data is recorded. The illustrated device 502 further includes a memory 507 to communicate with the processor, store computer-readable instructions to be executed by the processor to perform and control the sensing, pacing and other tasks performed by the CRM device. Additionally, CRT-related data including sensed and programmed parameters are stored in the memory for use to provide trends of the CRT delivery. The device 502 further includes a transceiver 508, such as telemetry circuitry, for use to communicate with external devices such as a programmer or a portable device.

The device 502 includes a number of interfaces for use to pace and/or sense a number of electrodes which are located on one or more leads that can be connected to the device. The illustrated device includes a number of interface channels 509, including interface 1, interface 2 and interface N. The number of interfaces, and the number of electrodes connected to the interfaces, is appropriate for the desired CRT application. The sensing and pacing configuration for the electrodes also can vary according to the CRT application. According to an embodiment, ventricular synchronization is provided using electrodes in the right and left ventricles, and atrial tracking capabilities is provided using an electrode in the right atrium. The illustrated device also includes sense circuitry 510, including an amplifier, for detecting sensed signals from the electrodes, and further includes pulse generator circuitry 511 for generating and delivering electrical pulses to an electrode 512. Appropriate protection of the circuitry is provided to allow both pacing and sensing. The illustrated device provides both pacing and sensing capabilities for each interface. However, other embodiments do not provide both pacing and sensing capabilities for each interface, because some CRT applications do not require each interface to perform both sensing and pacing functions.

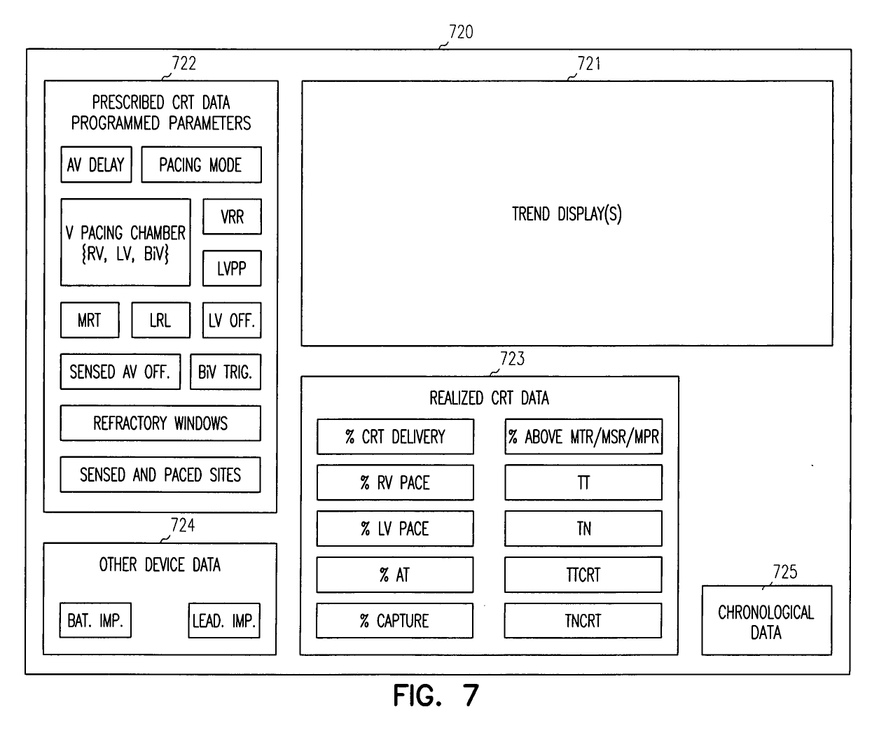

The memory 507 includes computer-readable instructions to be executed by the controller 505. According to various embodiments, the controller executes the instructions to record prescribed CRT data and time information in the memory. Examples of prescribed CRT data is illustrated and discussed with respect to FIG. 7. According to various embodiments, the controller executes the instructions to record realized CRT data and time information in the memory. Examples of realized CRT data are illustrated and discussed with respect to FIG. 7. According to various embodiments, the controller executes the instructions to record a pacing mode and time information in the memory. According to various embodiments, the controller executes the instructions to record an atrial tracking mode and time information in the memory. These embodiments are not mutually exclusive, as two or more of the above-identified embodiments can be combined.

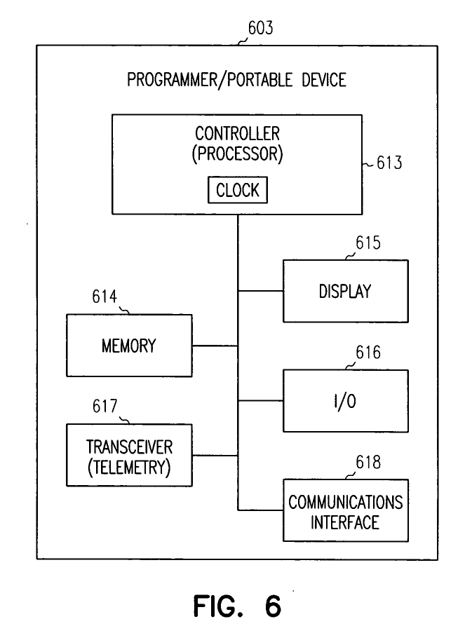

FIG. 6 illustrates a programmer or a portable APM device such as can be used in the system of FIG. 4, according to various embodiments of the present subject matter. To simply the discussion with respect to FIG. 6, the programmer or portable device is referred to simply as the programmer 603. The illustrated programmer includes a processor or controller 613 to communicate with a memory 614, a display 615, input/output devices 616 such as a speaker, mouse, keyboard and the like, a transceiver 617 such as telemetry circuitry, and a communication interface 618 such as a network adapter for wired or wireless communication. The recorded CRT-related data from the CRM device, which corresponds to the chronic, ambulatory status of the CRT, is capable of being stored in the memory of the programmer. In various embodiments, the programmer memory 614 stores computer-readable instructions to be executed by the processor to process the data into trends and to display the trended data on the programmer monitor. Furthermore, software allows a physician or clinician to select the desired trended data to be displayed on the programmer monitor. For example, an embodiment of the software provides a programmer display screen similar to the screen display illustrated in FIG. 7.

FIG. 7 illustrates a display such as can be projected on the programmer of FIG. 4, according to various embodiments of the present subject matter. The illustrated display 720 includes a trend display region 721 where CRT-related trends are capable of being displayed. Additionally, various flags and other warnings are capable of being displayed on the programmer. Other non-visual warnings can alternatively or additionally be provided using a speaker in the programmer.

The illustrated display 720 further includes a prescribed CRT data region 722, which includes buttons corresponding to examples of programmed parameters that can be selected for trending. The illustrated buttons are not intended to be an exclusive listing of all programmable parameters related to data. In the illustrated display, the prescribed CRT data region 722 includes buttons for AV DELAY, PACING MODE, V PACING CHAMBER{RV, LV, BiV□, VRR, LVPP, MRT, LRL, LV OFFSET, SENSED AV OFFSET, BiV TRIGGER, REFRACTORY WINDOWS, and SENSED AND PACED SITES. One of ordinary skill in the art will understand, upon reading and comprehending this disclosure, the meaning of these terms. In the interest of brevity, these programmed parameters related to prescribed CRT will not be discussed again here in view of the discussions of programmed parameters elsewhere in this disclosure.

The illustrated display 720 further includes a realized CRT data region 723, which includes buttons corresponding to examples of parameters that are indicative of the efficacy of the prescribed CRT in a chronic, ambulatory setting away from a clinical setting. The illustrated buttons are not intended to be an exclusive listing of all realized CRT data that are indicative of the prescribed CRT. In the illustrated display, the realized CRT data region 723 includes buttons for % CRT DELIVERY, % RV PACE, % LV PACE, % AT, % CAPTURE, % ABOVE MTR/MSR/MPR, TT (total time in tracking mode during period), TN (total time in non-tracking mode during period), TTCRT (total time CRT is successfully delivered in tracking mode), and TNCRT (total time CRT is successfully delivered in non-tracking mode). One of ordinary skill in the art will understand, upon reading and comprehending this disclosure, the meaning of these terms. In the interest of brevity, these realized parameters related to the efficacy of the prescribed CRT in a chronic, ambulatory setting away from a clinical setting will not be discussed again here in view of the discussions elsewhere in this disclosure.

The illustrated display 720 further includes an other device data region 724, which includes buttons corresponding to other device parameters that may potentially affect the successful delivery of CRT. For example, in the illustrated display, the other device data region includes buttons for battery impedance and lead impedance. The illustrated display 720 further includes a chronological data button 725 for use to select a desired time scale and duration for the displayed trends. For example, according to various embodiments, CRT-related parameter trends can be displayed for selected hour(s), day(s), and month(s). Thus, the physician or clinician is provided with desired information useful to assess the chronic, ambulatory status of the CRT delivery.

10. Methods

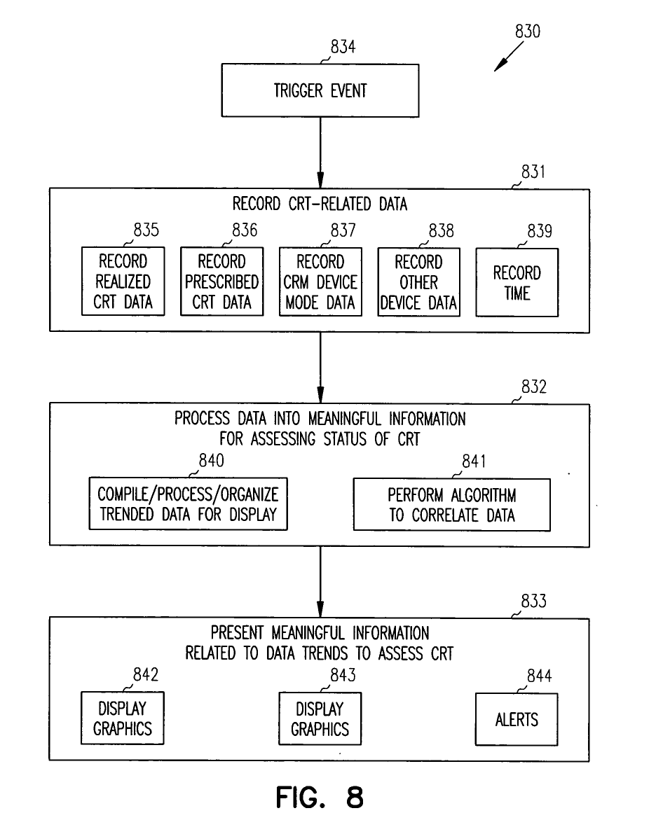

FIG. 8 is a flow diagram illustrating a trending method according to various embodiments of the present subject matter. This illustrated method 830 is not intended to be exclusive of other methods within the scope of the present subject matter. Those of ordinary skill in the art will understand, upon reading and comprehending this disclosure, other methods within the scope of the present subject matter.

The illustrated method for providing trended measurement of CRT includes recording CRT-related data at 831, processing data into meaningful information for assessing status of CRT at 832, and presenting meaningful information related to data trends such that a physician or clinician is capable of assessing the status of CRT at 833. According to an embodiment, a triggering event 834 controls when data is recorded at 831.

According to various embodiments, recording CRT-related data at 831 includes any one or any combination of the following: recording realized CRT-related data at 835, recording prescribed CRT-related data at 836, and recording CRM device mode data (e.g. pacing mode and the like) at 837. Other device data can be recorded at 838. Additionally, as represented at 839, the illustrated process includes recording the time when the CRT-related data is recorded. According to various embodiments, processing data into meaningful information for assessing chronic, ambulatory status of CRT at 832 includes any one or both of the following: compiling, processing and/or organizing trended data for display at 840, and performing or executing an algorithm to correlate the CRT-related data 841. In various embodiments, processing data into meaningful information is performed in the CRM device and/or the programmer. According to various embodiments, presenting meaningful information related to data trends such that a physician or clinician is capable of assessing the status of CRT at 833 includes displaying graphs of the trended data at 842, displaying a table that includes the trended data at 843, and/or providing alerts 844 such as messages that indicate proposed improvements to the prescribed CRT, flags, alarms and the like.

One of ordinary skill in the art will understand that, the modules and other circuitry shown and described herein can be implemented using software, hardware, and combinations of software and hardware. As such, the term module is intended to encompass software implementations, hardware implementations, and software and hardware implementations.

In various embodiments, the methods provided above are implemented as a computer data signal embodied in a carrier wave or propagated signal, that represents a sequence of instructions which, when executed by a processor cause the processor to perform the respective method. In various embodiments, methods provided above are implemented as a set of instructions contained on a computer-accessible medium capable of directing a processor to perform the respective method. In various embodiments, the medium is a magnetic medium, an electronic medium, or an optical medium.

11. Trend Example

FIG. 9 provides a graph 950 illustrating a trend example according to various embodiments of the present subject matter. An X axis 951 of the illustrated graph 950 represent the time during a day, and a Y axis 952 of the illustrated graph represents a quantity of time from 0 hours to 24 hours. The Y axis could also be represented as a percentage of time from 0% to 100% for the day.

TT₂₄ represents the total time that the CRM device is operating in a tracking mode over a 24-hour period, and TTCRT₂₄ represents the total time CRT therapy is successfully delivered while the CRM device is operating in a tracking mode over a 24-hour period. TN₂₄ represents the total time the CRM device is operating in a non-tracking mode over a 24-hour period, and TNCRT₂₄ represents the total time CRT therapy is successfully delivered while the CRM device is operating in a non-tracking mode over a 24-hour period.

A solid line 953 separates a lower region 954 and an upper region 955 of the graph. The lower region 954 represents the time that the CRM device is operating in a tracking mode TT24, and the upper region 955 represents the time that the CRM device is operating in a non-tracking mode TN₂₄. A first dotted line 956 separates the lower region 954 into a region 957 representing the time where the CRT is successfully delivered while operating in the tracking mode (TTCRT₂₄), and a region 958 representing the time where the CRT therapy is not successfully delivered while operating in the tracking mode. A second dotted line 959 separates the upper region 955 into a region 960 representing the time where the CRT therapy is successfully delivered while operating in the non-tracking mode (TNCRT₂₄), and a region 961 representing the time where the CRT is not successfully delivered while operating in the non-tracking mode. Thus, in the illustrated example, a clinician is provided with a visual representation of successful CRT delivery over the course of the day and for both tracking and non-tracking modes.

12. Concluding Remarks

Although specific embodiments have been illustrated and described herein, it will be appreciated by those of ordinary skill in the art that any arrangement which is calculated to achieve the same purpose may be substituted for the specific embodiment shown. This application is intended to cover adaptations or variations of the present subject matter. It is to be understood that the above description is intended to be illustrative, and not restrictive. Combinations of the above embodiments, and other embodiments will be apparent to those of skill in the art upon reviewing the above description. The scope of the present subject matter should be determined with reference to the appended claims, along with the full scope of equivalents to which such claims are entitled.

1. A method, comprising:
recording data related to a status of a prescribed cardiac resynchronization therapy (CRT) in a cardiac rhythm management (CRM) device;
processing the data into trended data useful for assessing the status of the prescribed CRT; and
presenting the trended data for use to assess the status of the prescribed CRT.

2. The method of claim 1, wherein recording data related to a status of a prescribed CRT in a CRM device includes recording realized CRT data.

3. The method of claim 2, wherein recording realized CRT data includes recording a value corresponding to CRT delivery, the value being at least one from the group consisting of a percentage value and an absolute value.

4. The method of claim 2, wherein recording realized CRT data includes recording a value corresponding to ventricular pacing, the value being at least one from the group consisting of a percentage value and an absolute value, the ventricular pacing being at least one from a group consisting of right ventricular pacing (RV PACE) and left ventricular pacing (LV PACE).

5. The method of claim 2, wherein recording realized CRT data includes recording a value corresponding to atrial tachycardia (AT), the value being at least one from the group consisting of a percentage value and an absolute value.

6. The method of claim 2, wherein recording realized CRT data includes recording a value corresponding to capture, the value being at least one value from the group of values consisting of a percentage value and an absolute value.

7. The method of claim 2, wherein recording realized CRT data includes recording a value corresponding to a value above a programmed rate, the value being at least one from the group consisting of a percentage value and an absolute value, the programmed rate being at least one from the group consisting of a programmed maximum tracking rate (MTR), a programmed maximum sensing rate (MSR), and a programmed maximum pacing rate (MPR).

8. The method of claim 2, wherein recording realized CRT data includes recording a value corresponding to a mode of operation, the value being at least one from the group consisting of a percentage value and an absolute value, the mode being at least one from the group consisting of a tracking mode (TT) of operation and a non-tracking mode (TN) of operation.

9. The method of claim 2, wherein recording realized CRT data includes recording a value corresponding to a CRT delivery results, the value being at least one from the group consisting of a percentage value and an absolute value, the CRT delivery results being at least one from the group consisting of CRT therapy that was successfully delivered (TTCRT) and CRT therapy that was not successfully delivered (TNCRT).

10. The method of claim 1, wherein recording data related to a status of a prescribed CRT in a CRM device includes recording prescribed CRT data corresponding to programmed CRT-related parameters.

11. The method of claim 1, wherein recording data related to a status of a prescribed CRT in a CRM device includes recording a CRM device operating mode.

12. The method of claim 11, wherein recording a CRM device operating mode includes recording data to permit association between recorded data and a pacing mode of the CRM device when the data is recorded.

13. The method of claim 1, wherein recording data related to a status of a prescribed CRT in a CRM device includes recording other CRM device data capable of affecting the status of the prescribed CRT in the CRM device.

14. The method of claim 1, wherein recording data related to a status of a prescribed CRT in a CRM device includes recording a time associated with recording at least one of realized CRT data, prescribed CRT data, and a CRM device operating mode.

15. The method of claim 1, wherein processing the data into trended data useful for assessing the status of the prescribed CRT includes processing the trended data for displaying a representation of the trended data.

16. The method of claim 1, wherein processing the data into trended data useful for assessing the status of the prescribed CRT includes executing an algorithm on the data to assist with assessing the status of the CRT.

17. The method of claim 1, further comprising using the status of the prescribed CRT to assist with determining an adjustment to improve the prescribed CRT.

18. The method of claim 17, wherein presenting the trended data for use to assess the status of the prescribed CRT includes displaying graphs of the trended data.

19. The method of claim 17, wherein presenting the trended data for use to assess the status of the prescribed CRT includes displaying a table containing the trended data.

20. The method of claim 17, wherein presenting the trended data for use to assess the status of the prescribed CRT includes providing an alert corresponding to the status of the prescribed CRT.

21. The method of claim 1, wherein the status of the prescribed CRT includes a chronic, ambulatory status.

22. The method of claim 1, further comprising initiating a trigger, wherein data related to the status of the prescribed CRT in the CRM device is recorded after initiating the trigger.

23. The method of claim 1, wherein processing the data into trended data includes trending N samples per unit time.

24. The method of claim 1, wherein processing the data into trended data includes trending N samples per unit time until a predetermined change occurs in delivered CRT, and then trending M samples per unit time.

25. The method of claim 1, wherein processing the data into trended data includes trending N samples per unit time until a predetermined threshold is reached related to delivered CRT, and then trending M samples per unit time.

26. The method of claim 1, wherein processing the data into trended data includes trending N samples per unit time until a predetermined event occurs, and then trending M samples per unit time.

27. The method of claim 1, wherein processing the data into trended data includes trending M samples per unit time after initiation of a trigger selected from a group consisting of: a predetermined change in delivered CRT, a predetermined threshold related to delivered CRT, and a predetermined event.

28. The method of claim 1, wherein processing the data into trended data includes:
trending a first parameter before a trigger selected from a group consisting of a predetermined change in delivered CRT, a predetermined threshold related to delivered CRT, and a predetermined event; and
trending a second parameter after the trigger.

29. An implantable cardiac rhythm management (CRM) device, comprising:
a plurality of interface channels to interface with a plurality of electrodes on at least one lead, wherein the plurality of interface channels are adapted to deliver pacing pulses to at least one of the plurality of electrodes and to receive sensed cardiac signals from at least one of the plurality of electrodes as part of a prescribed cardiac resynchronization therapy (CRT);
a memory embedded with computer instructions;
a controller to communicate with the plurality of interface channels and with the memory, the controller to control delivery of the pacing pulses, to process the sensed signals, and to record CRT-related data relevant to a status of the prescribed CRT to the memory; and
a communication circuit to transmit the CRT-related data to an external device for presentation of data trends useful to assess the status of the prescribed CRT.

30. The device of claim 29, wherein the data relevant to a status of the prescribed CRT includes a chronic, ambulatory data.

31. The device of claim 29, wherein the plurality of interface channels include:
a right ventricle (RV) interface channel for use in sensing and pacing a right ventricle of a heart;
a left ventricle (LV) interface channel for use in sensing and pacing a left ventricle of a heart; and
a right atrium (RA) interface channel for use in sensing and pacing an atrium of the heart.

32. The device of claim 29, wherein the controller is adapted to record prescribed CRT data and time information in the memory.

33. The device of claim 29, wherein the controller is adapted to record realized CRT data and time information in the memory.

34. The device of claim 29, wherein the controller is adapted to record a pacing mode and time information in the memory.

35. The device of claim 34, wherein the controller is adapted to record when the device is operating in an atrial tracking mode to the memory.

36. The device of claim 29, wherein the controller is adapted to trend samples of CRT-related data relevant to the status of the prescribed CRT to the memory, including to trend N samples per unit time.

37. The device of claim 29, wherein the controller is adapted to trend samples of CRT-related data relevant to the status of the prescribed CRT to the memory, including to trend N samples per unit time until a predetermined change occurs in delivered CRT, and then trend M samples per unit time.

38. The device of claim 29, wherein the controller is adapted to trend samples of CRT-related data relevant to the status of the prescribed CRT to the memory, including to trend N samples per unit time until a predetermined threshold is reached related to delivered CRT, and then trend M samples per unit time.

39. The device of claim 29, wherein the controller is adapted to trend samples of CRT-related data relevant to the status of the prescribed CRT to the memory, including to trend N samples per unit time until a predetermined event occurs, and then trend M samples per unit time.

40. The device of claim 29, wherein the controller is adapted to trend samples of CRT-related data relevant to the status of the prescribed CRT to the memory, including to trend M samples per unit time after initiation of a trigger selected from a group consisting of: a predetermined change in delivered CRT, a predetermined threshold related to delivered CRT, and a predetermined event.

41. The device of claim 29, wherein the controller is adapted to trend samples of CRT-related data relevant to the status of the prescribed CRT to the memory, including to trend a first parameter before a trigger selected from a group consisting of a predetermined change in delivered CRT, a predetermined threshold related to delivered CRT, and a predetermined event, and to trend a second parameter after the trigger.

42. The device of claim 41, wherein the CRT-related data includes a value corresponding to CRT delivery, the value being at least one from the group consisting of a percentage value and an absolute value.

43. The device of claim 41, wherein the CRT-related data includes a value corresponding to ventricular pacing, the value being at least one from the group consisting of a percentage value and an absolute value, the ventricular pacing being at least one from a group consisting of right ventricular pacing (RV PACE) and left ventricular pacing (LV PACE).

44. The device of claim 41, wherein the CRT-related data includes a value corresponding to atrial tachycardia (AT), the value being at least one from the group consisting of a percentage value and an absolute value.

45. The device of claim 41, wherein the CRT-related data includes a value corresponding to capture, the value being at least one value from the group of values consisting of a percentage value and an absolute value.

46. The device of claim 41, wherein the CRT-related data includes a value corresponding to a value above a programmed rate, the value being at least one from the group consisting of a percentage value and an absolute value, the programmed rate being at least one from the group consisting of a programmed maximum tracking rate (MTR), a programmed maximum sensing rate (MSR), and a programmed maximum pacing rate (MPR).

47. The device of claim 41, wherein the CRT-related data includes a value corresponding to a mode of operation, the value being at least one from the group consisting of a percentage value and an absolute value, the mode being at least one from the group consisting of a tracking mode (TT) of operation and a non-tracking mode (TN) of operation.

48. The device of claim 41, wherein the CRT-related data includes a value corresponding to a CRT delivery results, the value being at least one from the group consisting of a percentage value and an absolute value, the CRT delivery results being at least one from the group consisting of CRT therapy that was successfully delivered (TTCRT) and CRT therapy that was not successfully delivered (TNCRT).

49. A system, comprising:
an implantable cardiac rhythm (CRM) device to perform a prescribed cardiac resynchronization therapy (CRT), the CRM device including:
a set of interface channels to provide a prescribed cardiac resynchronization therapy (CRT), wherein at least one of the channels is adapted to deliver pacing pulses to at least one of a plurality of electrodes and at least one of the channels is adapted to receive sensed cardiac signals from at least one of the plurality of electrodes;
a memory embedded with controller instructions;
a controller to communicate with the set of interface channels and the memory, the controller to execute the controller instructions to control delivery of the pacing pulses, to process sensed cardiac signals, and to record CRT-related data relevant to a status of the prescribed CRT to the memory of the CRM device; and
a communication circuit to communicate with the controller and to transmit and receive wireless communication signals; and
a programmer to program the CRM device to provide the prescribed CRT, the programmer including:
a memory embedded with controller instructions;
a controller to communicate with the memory and execute the controller instructions;
a communication circuit to communicate with the controller and to transmit and receive wireless communication signals such that the programmer is capable of wirelessly communicating with the CRM device, and such that the CRT-related data is communicated from the memory of the CRM device to the memory of the programmer; and
a monitor to communicate with the controller to display information corresponding to trended data samples indicative of the status of the prescribed CRT.

50. The system of claim 49, wherein the status of the prescribed CRT includes a chronic, ambulatory status.

51. The system of claim 49, wherein the memory of the CRM device includes controller instructions to be executed by the controller of the CRM device to trend the data samples.

52. The system of claim 49, wherein the memory of the programmer includes controller instructions to be executed by the controller of the programmer to trend the data samples.

53. The system of claim 49, wherein the information displayed on the monitor includes at least one of:
a graph of the trended data samples;
a table containing the trended data samples; and
an alert corresponding to the status of the prescribed CRT.

54. A system, comprising:
an implantable cardiac rhythm (CRM) device to perform a prescribed cardiac resynchronization therapy (CRT), the CRM device including:
means for delivering pacing pulses to at least one of a plurality of electrodes;
means for receiving sensed cardiac signals from at least one of the plurality of electrodes;
means for controlling delivery of the pacing pulses and processing the sensed cardiac signals to perform the prescribed CRT;
means for recording CRT-related data corresponding to a status of the prescribed CRT; and
means to transmit and receive wireless communication signals; and
a programmer to program the CRM device to provide the prescribed CRT, the programmer including:
means to transmit and receive wireless communication signals such that the programmer is capable of wirelessly communicating with the CRM device; and
means to display information corresponding to trended data indicative of the status of the prescribed CRT.

55. The system of claim 54, wherein the status of the prescribed CRT includes a chronic, ambulatory status of the prescribed CRT.

56. The system of claim 54, wherein the means to display information includes a graph of the trended data.

57. The system of claim 54, wherein the means to display information includes a table of the trended data.

58. The system of claim 54, wherein the CRT-related data includes prescribed CRT data.

59. The system of claim 54, wherein the CRT-related data includes realized CRT data.

60. The system of claim 54, wherein the CRM device further includes means to detect a trigger, and means to trend data samples based on the trigger.
